(19) United States
(12) Patent Application Publication
(10) Pub. No.: US 2006/0177405 A1
(43) Pub. Date: Aug. 10, 2006

(54) Moisturizing compositions

(76) Inventors: Maureen Sullivan Morrissey, Belmont, MA (US); Alexander Novikov, Framingham, MA (US); Janet Kelley O'Grady, Westwood, MA (US); Stephen Thong, Pennington, NJ (US)

Correspondence Address: FISH & RICHARDSON P.C., P.O. BOX 1022, MINNEAPOLIS, MN, 55440-1022, US

(21) Appl. No.: 11/051,937

(22) Filed: Feb. 4, 2005

(51) Int. Cl. A61K 8/55

(52) U.S. Cl. 424/70.21

(57) ABSTRACT

A moisturizing composition includes water, a phospholipid, a humectant, and an emollient. The emollient is a fatty ester that has the formula R₁—COO—R, wherein R is from an alkyl moiety or a polyol moiety, R₁ is from an acyl moiety of a mono- or di-carboxy fatty acid, and R and R₁ each have a carbon chain length of up to 10 carbon atoms.

### TECHNICAL FIELD

This invention relates to moisturizing compositions for use in skin care, such as moisturizing aftershave balms or lotions.

### BACKGROUND

Aftershave balms or lotions are typically oil-in-water or water-in-oil emulsions that can moisturize skin and/or soothe irritated skin (e.g., razor burned skin). While water-in-oil emulsions can be more effective at moisturizing than oil-in-water emulsions, water-in-oil emulsions can be greasier and/or oilier than oil-in-water emulsions. Furthermore, some water-in-oil emulsions can be relatively tacky, and can leave a tacky residue on the skin.

### SUMMARY

The invention relates to moisturizing compositions, such as balms or lotions. When applied to the skin, the moisturizing compositions can hydrate the skin and make it feel smooth, without making the skin feel especially oily and/or greasy. The moisturizing compositions are oil-in-water emulsions than can include one or more phospholipids (e.g., phosphatidylcholine), emollients, and humectants.

In one aspect, the invention features a moisturizing composition that includes at least about 70% water, about 0.05% to about 1.5% of a phospholipid, about 0.5% to about 15% of a humectant, and about 0. 1% to about 10% of an emollient. The humectant includes a polyol with a carbon chain length of up to eight carbon atoms. The emollient includes a fatty ester. The moisturizing composition is in the form of a balm or lotion.

In another aspect, the invention features a method of moisturizing skin, the method including applying to the skin a moisturizing composition having the features described above.

In a further aspect, the invention features a moisturizing composition including water, phosphatidylcholine, pentylene glycol, and isononyl isononanoate. The moisturizing composition is in the form of a balm or lotion.

The method can include shaving the skin prior to applying the moisturizing composition to the skin (e.g., such that the moisturizing composition serves as an aftershave balm or an aftershave lotion).

Some embodiments can include one or more of the following advantages.

The moisturizing composition can be highly effective at moisturizing skin-while also not feeling greasy and/or oily on the skin. The moisturizing composition can be relatively non-tacky, such that it will not leave a tacky residue on the skin when applied. Application of the moisturizing composition can leave skin feeling smooth. The moisturizing composition can remain on the skin for a relatively long period of time after application. The moisturizing composition can be used to soothe irritated skin, such as skin that is irritated as the result of a razor burn. In some instances, the moisturizing composition can help to rehydrate dry skin and, for example, bring the skin back to its natural moisturization level.

Other features and advantages of the invention will be apparent from the description and the claims.

### DETAILED DESCRIPTION

A preferred moisturizing composition includes water, a phospholipid, a humectant that includes a polyol, and an emollient that includes a fatty ester. Generally, the moisturizing composition is an oil-in-water emulsion in the form or a balm or a lotion that can be used to moisturize the skin (e.g., after shaving).

Preferably, the moisturizing composition has a viscosity of about 2,000 centipoise to about 30,000 centipoise, more preferably about 7,000 centipoise to about 20,000 centipoise (RVT viscometer, spindle no. 5, 20 rpm, 4 revolutions, @ 26-27 C), a pH of 5 to 7, more preferably 5 to 6, and a density of about 0.9 gram per cubic centimeter to about one gram per cubic centimeter, more preferably about 0.93 gram per cubic centimeter to about 0.97 gram per cubic centimeter.

The emollient provides the moisturizing composition with a softening and/or soothing effect on skin. For example, the emollient can contribute to the ability of the moisturizing composition to soothe irritated skin, such as skin that has been irritated by razor bum. Furthermore, the emollient can cause the moisturizing composition to form a barrier layer on the skin that can serve to minimize water loss. Preferred moisturizing compositions include about 0.1% to about 10%, more preferably about 0.1% to about 5%, of the emollient.

The emollient is preferably a linear or branched ester with the formula R₁—COO—R. R and R₁ each have a carbon chain length of up to 10 carbon atoms, and preferably have a carbon chain length of from three carbon atoms to 10 carbon atoms. R and R₁ may have the same carbon chain length, or may have different carbon chain lengths. R may be from an alkyl moiety or a polyol moiety. Suitable alkyl moieties may include, for example, ethylhexyl, isononyl, isodecyl, isopropyl, and caprylyl. Suitable polyol moieties may include, for example, glycerol, neopentyl glycol, butylene glycol, pentylene glycol, sorbitol, hexylene glycol, and caprylyl glycol. R₁ can be from an acyl moiety of a mono- or di-carboxy fatty acid. Suitable mono-acyl moieties may include heptanoyl (heptanoic acid), capriloyl (caprylic acid), isooctanoyl (isooctanoic acid), isononanoyl (isononanoic acid) or neopentanoyl (neopentanoic acid). Suitable acyl moieties for di-carboxy acids may include adipoyl (adipic acid) or sebacoyl (sebacic acid).

A preferred emollient is isononyl isononanoate. Other suitable emollients include isooctyl isooctanoate, diisopropyl adipate, glycerin heptanoate, ethylhexyl caprylate, diisopropyl sebacate, neopentylglycol diheptanoate, and isodecyl neopentanoate.

Preferably, the emollient is a liquid at room temperature (25° C.). Waxes and oils that are solid at room temperature are generally not preferred.

The humectant contributes to the moisturizing properties of the moisturizing composition. For example, the humectant can attract water to the skin, thereby counteracting the effects of water loss by the skin. Preferred moisturizing compositions include about 0.5% to about 15%, more preferably about 2% to about 6%, of the humectant.

The humectant is preferably a polyol with a carbon chain length of up to eight carbon atoms (e.g., from three carbon atoms to eight carbon atoms) and three to six hydroxyl groups. The polyol typically has a relatively low molecular weight (e.g., a molecular weight of less than about 146). Preferably, the humectant is pentylene glycol. Examples of other suitable humectants include glycerol, butylene glycol, sorbitol, hexylene glycol, and caprylyl glycol.

The phospholipid helps to replenish the natural level of moisturization of the skin. Preferably, the moisturizing composition includes about 0.05% to about 1.5%, more preferably about 0.05% to about 1%, of the phospholipid. The phospholipid can be zwitterionic, and can have a purity of greater than about 80%, more preferably greater than 90%. Preferably, the phospholipid is phosphatidylcholine.

Water is the major component of the moisturizing composition, and typically is used in sufficient quantities to provide an oil-in-water emulsion with a suitable viscosity to be used as an aftershave balm or lotion. Water is added in a sufficient quantity (q.s.) to bring the total of all components to 100%. The moisturizing composition can include at least about 70%, preferably about 75% to about 95%, and more preferably about 80% to about 90% water.

In some embodiments, the moisturizing composition can include additional ingredients. Examples of other suitable ingredients include preservatives, fragrances, skin care actives, occlusive agents, emulsifiers, detackifying agents, thickeners, gellants, neutralizers, UV stabilizers, colorants, absorbents, essential oils, and the like. Suitable skin care actives for use in the moisturizing composition include oil- and water-soluble vitamins (e.g., vitamin A, vitamin E, vitamin C, vitamin B3) and anti-irritants (e.g., bisabolol, aloe, glycyrrhyzinic acid). A suitable absorbent for use in the moisturizing composition is starch, which can help to absorb oil from the skin. Another suitable absorbent is oat flour. In certain embodiments, the thickener can be a water-soluble thickener (e.g., polyacrylamide, polysaccharide), and/or an oil-soluble thickener (e.g., pentaerythrityl tetrastearate).

The moisturizing composition can, for example, include one or more of the following ingredients in the following amounts: up to about 7.5% (e.g., up to about 3.5%) of a detackifying agent (e.g., aluminum starch octenylsuccinate); up to about 6% (e.g., up to about 2%, up to about 1%) of an occlusive agent (e.g., cetyl palmitate); up to about 5.5% (e.g., up to about 1.5%, up to about 1%) of an emulsifier (e.g., PEG-30 lanolin, propylene glycol monostearate); up to about 2% (e.g., up to about 1%) of a gellant (e.g., carbomer); up to about 6% (e.g., up to about 0.6%) of vitamins and/or skin care actives; and/or up to about 0.5% (e.g., up to about 0.1%) of a UV stabilizer (e.g., benzophenone-4). In some embodiments, the moisturizing composition can include more than one type of a particular ingredient. For example, the moisturizing composition can include two different types of occlusive agents (e.g., mineral oil and cetyl palmitate).

The moisturizing compositions described herein are generally applied topically to the human skin. While the moisturizing compositions can be applied to the face, application is not necessarily limited to the face. For example, in some embodiments, the moisturizing compositions can be applied to the legs, arms, back, bikini area, and/or any other area of skin. While application of the moisturizing compositions after shaving has been described, it is not essential that the moisturizing compositions be applied to shaved skin. For example, the moisturizing compositions can be applied to soothe unshaved skin. The moisturizing compositions can be applied once a day or multiple times a day, and/or can be applied daily, weekly, monthly, or yearly, at regular or irregular intervals.

Because they are provided in the form of balms and lotions, the moisturizing compositions are generally substantially free of propellant. For example, the moisturizing compositions generally include less than about 0.5% propellant, and preferably less than 0.1% propellant. However, in some cases, e.g., in the case of microemulsions, it may be desirable to include a hydrocarbon propellant to function as a blowing agent.

The moisturizing compositions described herein can be formed using any suitable methods and/or manufacturing processes. An example of a suitable process is as follows.

First, the water phase and the oil phase of the moisturizing composition, along with a gellant phase, are prepared separately. The water phase includes water, humectant, water-soluble UV stabilizer(s), and a small amount of the neutralizer. The oil phase includes the oil-soluble/dispersible components, such as the emollient. The water phase and the oil phase are separately heated to 75° C. The gellant phase, which includes gellant and water, is prepared in this example by dispersing the gellant in water and homogenizing the resulting dispersion.

Next, the oil phase and the water phase are combined at about 75° C. The resulting mixture of the oil phase and the water phase is agitated thoroughly for about 30 minutes, and then cooled to a temperature of about 65° C. Next, the phospholipid is added to the mixture, and the mixture is cooled to a temperature of about 45° C. Thereafter, starch can be added to the mixture, and the mixture continues to cool to about 35° C. Once the mixture is at about 35° C., preservatives are added to the mixture. Any remaining ingredients that are temperature-sensitive (e.g., preservatives, fragrance, skin care actives and oat flour) can be added when the mixture is at a temperature of about 30° C. or less. The gellant phase is then added into the mixture and neutralized with more of the neutralizer (sodium hydroxide). Neutralizer is added to the entire batch until the desired target pH is reached (in this example, 5 to 6). This is the last step in the process; at the time of neutralization the gellant phase, in its un-neutralized form, has been combined with the water and oil phases.

The following example is intended to be illustrative and non-limiting.

### EXAMPLE

A moisturizing composition was prepared using the above-described process. The components of the composition, as well as their respective weight percentages in the composition, are shown in Table 1.

TABLE 1

| | |
|---|---|
| Component | Weight Percent |

| | |
|---|---|
| Water | q.s. to 100 |
| Aluminum Starch | 2.30 |
| Octenylsuccinate | |
| Pentylene Glycol | 4.00 |
| Isononyl Isononanoate | 1.00 |
| Myristyl Propionate | 1.00 |
| Cetyl Palmitate | 0.50 |
| PEG-30 Lanolin | 0.50 |
| Carbomer (Carbopol 5984) | 0.35 |
| Oat Flour | 0.30 |
| Phospholipid | 0.20 |
| (Phospholipon 90 NG) | |
| Skin Care Active | 0.80 |
| Sodium Hydroxide | q.s. to pH of 5.5 ± 0.5 |
| Propylene Glycol | 0.10 |
| Monostearate | |
| Benzophenone-4 | 0.02 |

The composition described in Table 1 exhibited good moisturizing properties, including preventing irritation, protecting from razor burn, and leaving skin soft.

As used in this application (unless indicated otherwise), all percentages are by weight on a solids basis.

Other embodiments are within the scope of the following claims.

What is claimed is:

1. A moisturizing composition, comprising:
at least about 70% water;
about 0.05% to about 1.5% of a phospholipid;
about 0.5% to about 15% of a humectant comprising a polyol with a carbon chain length of up to eight carbon atoms; and
about 0.1% to about 10% of an emollient comprising a fatty ester having the formula R₁—COO—R, wherein R is from an alkyl moiety or a polyol moiety, R1 is from an acyl moiety of a mono- or di-carboxy fatty acid, and R and R₁ each have a carbon chain length of up to 10 carbon atoms,
wherein the moisturizing composition is in the form of a balm or lotion.

2. The moisturizing composition of claim 1, wherein the phospholipid comprises phosphatidylcholine.

3. The moisturizing composition of claim 2, wherein the moisturizing composition comprises about 0.05% to about 1% of the phospholipid.

4. The moisturizing composition of claim 1, wherein the moisturizing composition comprises about 2% to about 6% of the humectant.

5. The moisturizing composition of claim 4, wherein the moisturizing composition comprises about 0.1% to about 5% of the emollient.

6. The moisturizing composition of claim 1, wherein the phospholipid is zwitterionic.

7. The moisturizing composition of claim 1, wherein the phospholipid has a purity of greater than about 80%.

8. The moisturizing composition of claim 7, wherein the phosphatidylcholine has a purity of greater than about 90%.

9. The moisturizing composition of claim 4, wherein the humectant comprises a polyol selected from the group consisting of glycerol, butylene glycol, sorbitol, hexylene glycol, caprylyl glycol, and neopentyl glycol.

10. The moisturizing composition of claim 1, wherein the humectant comprises pentylene glycol.

11. - The moisturizing composition of claim 1, wherein the emollient comprises a branched fatty ester.

12. The moisturizing composition of claim 1, wherein the emollient comprises a linear fatty ester.

13. The moisturizing composition of claim 1, wherein the emollient comprises a fatty ester that is a liquid at 25° C.

14. The moisturizing composition of claim 1, wherein the emollient comprises isononyl isononanoate.

15. The moisturizing composition of claim 1, wherein the emollient comprises a fatty ester selected from the group consisting of isooctyl isooctanoate, diisopropyl adipate, glyceryl heptanoate, diisopropyl sebacate, neopentylglycol diheptanoate, isodecyl neopentanoate, and ethylhexyl caprylate.

16. A method of moisturizing skin, the method comprising:
applying a moisturizing composition to the skin, wherein the moisturizing composition is in the form of a balm or a lotion and comprises:
at least about 70% water;
about 0.05% to about 1.5% of a phospholipid;
about 0.5% to about 15% of a humectant comprising a polyol with a carbon chain length of up to eight carbon atoms; and
about 0.1% to about 10% of an emollient comprising a fatty ester having the formula R₁—COO—R, wherein R is from an alkyl moiety or a polyol moiety, R₁ is from an acyl moiety of a mono- or di-carboxy fatty acid, and R and R₁ each have a carbon chain length of up to 10 carbon atoms.

17. The method of claim 16, further comprising shaving the skin prior to applying the moisturizing composition to the skin.

18. A moisturizing composition, comprising:
water;
a phosphatidylcholine;
pentylene glycol; and
isononyl isononanoate,
wherein the moisturizing composition is in the form of a balm or lotion.

19. The moisturizing composition of claim 18, wherein the moisturizing composition comprises at least about 70% water.

20. The moisturizing composition of claim 18, wherein the moisturizing composition comprises about 0.05% to about 1.5% of the phosphatidylcholine.

21. The moisturizing composition of claim 20, wherein the moisturizing composition comprises about 0.5% to about 15% of the pentylene glycol.

22. The moisturizing composition of claim 21, wherein the moisturizing composition comprises about 0.1% to about 10% of the isononyl isononanoate.
